(19) United States
(12) Patent Application Publication
(10) Pub. No.: US 2012/0116356 A1
(43) Pub. Date: May 10, 2012

(54) Cleansing Device

(76) Inventor: Felishia Davenport, Baton Rough, LA (US)

(21) Appl. No.: 12/941,036

(22) Filed: Nov. 6, 2010

(51) Int. Cl. A61B 19/00; B23P 17/04

(52) U.S. Cl. 604/540; 29/428

(57) ABSTRACT

A cleansing device for cleaning a body cavity is provided to a user. The cleansing device comprises an inner membrane fabricated from an elastomeric material and an outer membrane fabricated from a hypoallergenic material. The inner surface of the inner membrane defines a cavity that conforms to and accommodates the user's finger. The inner membrane is in contact with and enveloped by the outer membrane to form the cleansing device. The user inserts the finger into the cavity of the cleansing device, where the inner surface of the inner membrane contacts the inserted finger, and the outer surface of the outer membrane is exposed for cleaning the body cavity. The user introduces the enclosed finger into the body cavity for cleaning. A string attached between the outer surface of the inner membrane and the inner surface of the outer membrane enables removal of the cleansing device from the user's finger.

### BACKGROUND

A body cavity, for example, the vagina is a tubular cavity lined with a mucous membrane. The environment in the vaginal canal is kept moist and alkaline by multiple vaginal secretions. In a healthy vagina, the secretions are clean, mucoid, and have good lubricating properties. However, debris in the form of desquamating epithelial cells, blood left from menstrual flow, and deposition of seminal fluid accumulate in the vaginal canal, thereby rendering the vaginal canal an incubator for many pathogenic and non-pathogenic bacteria. The vaginal canal is therefore prone to infections. Moreover, the debris, at times, emits a foul odor. Therefore, there is a need for a personal hygiene device that can be used for cleaning and refreshing the vaginal canal to prevent offensive odors and to combat infections in the vaginal canal.

Conventional devices for cleaning a vaginal canal include applicators and foam based cleaning solutions. The applicators are often difficult to use, rigid, and inflexible, thereby causing pain and discomfort during vaginal insertion. The foam based cleaning solutions tend to cause irritation in the vaginal canal. In order to minimize pain during insertion and to avoid causing irritation to the vaginal canal, there is a need for a cleansing device that is safe, effective, and convenient to use. Furthermore, there is a need for a cleansing device that is portable, easy to construct and use, and easily disposable.

Hence, there is a long felt but unresolved need for a cleansing device that enables a user to clean a body cavity, for example, the vaginal canal both safely and effectively.

### SUMMARY OF THE INVENTION

This summary is provided to introduce a selection of concepts in a simplified form that are further described in the detailed description of the invention. This summary is not intended to identify key or essential inventive concepts of the claimed subject matter, nor is it intended for determining the scope of the claimed subject matter.

The device disclosed herein addresses the above stated need for an easily constructible, easy to use, portable, and easily disposable device for cleaning a body cavity of a user. The body cavity is, for example, a vaginal canal of the user. The device for cleaning the body cavity of the user is herein referred to as a “cleansing device”. The cleansing device disclosed herein comprises an inner membrane and an outer membrane. The outer membrane is fabricated from a non-bulky hypoallergenic material, for example, cotton, microfiber, feather down, etc. The inner membrane is fabricated from an elastomeric material, for example, nitrile, latex, cyanamidite, peridine, amine, etc., and any combination thereof.

Each of the inner membrane and the outer membrane defines an inner surface and an outer surface. The inner surface of the inner membrane defines a cavity configured to conform to and accommodate a finger of the user. The inner membrane is in contact with and enveloped by the outer membrane to form the cleansing device. The user inserts the finger into the cavity of the cleansing device, where the inner surface of the inner membrane contacts the user's finger, and the outer surface of the outer membrane is exposed for cleaning the body cavity. The cleansing device encloses the user's inserted finger and the user introduces the enclosed finger into the body cavity for cleaning the body cavity. The outer surface of the outer membrane contacts the surface of the body cavity to be cleaned, when the user introduces the enclosed finger into the body cavity. In an embodiment, the user soaks the cleansing device enclosing the user's finger in a sterile solution, for example, water. The soaked device grips the user's finger and prevents slippage of the cleansing device from the user's finger during cleaning of the body cavity. The soaked device improves the grip of the soaked device on the user's finger.

The user cleans the body cavity using the exposed outer surface of the outer membrane of the cleansing device by moving the enclosed finger in horizontal, vertical, and circular directions along an inner wall of the body cavity. The user rinses the sanitary cleansing device in a sterile solution, for example, water, after retraction of the enclosed finger from the body cavity and prior to reintroduction of the enclosed finger into the body cavity. The cleansing device that encloses the user's finger grips the user's finger and prevents slippage of the cleansing device from the user's finger during the cleaning of the body cavity.

In an embodiment, the sanitary cleansing device further comprises a string attached between the outer surface of the inner membrane and the inner surface of the outer membrane for enabling removal of the cleansing device from the user's finger. The string is made of a hypoallergenic material, for example, cotton. The cleansing device is removed from the user's finger by outwardly pulling the string such that the outer surface of the outer membrane used for cleaning the body cavity folds inwardly and the inner surface of the inner membrane is exposed outwardly for ensuring safe and hygienic disposal of the cleansing device.

In another embodiment, the cleansing device is cleaned by soaking the cleansing device in a sterile solution, for example, water. In another embodiment, the inner surface of the inner membrane is configured for gripping the user's finger. For example, the inner surface of the inner membrane has a ribbed structure that provides additional grip between the user's finger and the inner surface of the inner membrane.

In another embodiment, the cleansing device further comprises a covering member that covers the outer membrane of the cleansing device. The covering member contains one or more permeable fluids. The covering member containing the permeable fluids envelopes the outer membrane of the cleansing device and releases the permeable fluids over the outer surface of the outer membrane. The permeable fluids comprise, for example, one or more of a medicating element, a germicide, a perfuming element, a lubricant, etc., and any combination thereof. In another embodiment, the outer membrane of the cleansing device is a moisture laden outer membrane for providing smooth contact between the outer surface of the outer membrane and the body cavity.

In another embodiment, the cleansing device further comprises a pleated layer enveloping the outer membrane of the cleansing device. The pleated layer is made of an adsorbent material, for example, a sponge, that contains a sanitizing fluid for disinfecting and cleaning the body cavity. In another embodiment, the outer membrane is configured with pleats that contain a sanitizing fluid for disinfecting and cleaning the body cavity.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and components disclosed herein.

FIG. 1 exemplarily illustrates a cleansing device for cleaning a body cavity.

FIG. 2 exemplarily illustrates an inner membrane and an outer membrane used for constructing the cleansing device.

FIG. 3 exemplarily illustrates a soakable cleansing device.

FIG. 4 exemplarily illustrates introduction of a user's finger enclosed by the cleansing device into a body cavity.

FIG. 5 exemplarily illustrates removal of the cleansing device from the user's finger by a string.

FIG. 6 exemplarily illustrates an embodiment of the cleansing device, showing a covering member covering the outer membrane of the cleansing device.

FIG. 7 exemplarily illustrates an embodiment of the cleansing device, showing a moisture laden outer membrane of the cleansing device.

FIG. 8 exemplarily illustrates an embodiment of the cleansing device, showing the configuration of the inner membrane of the cleansing device for improving grip of the cleansing device on the user's finger.

FIG. 9 exemplarily illustrates an embodiment of the cleansing device, showing the thicknesses of the inner membrane and the outer membrane of the cleansing device for improving grip of the cleansing device on the user's finger.

FIG. 10A exemplarily illustrates an embodiment of the cleansing device, showing a pleated layer enveloping the outer membrane of the cleansing device.

FIG. 10B exemplarily illustrates an embodiment of the cleansing device, showing the outer membrane of the cleansing device configured with pleats.

FIG. 11 illustrates a method for cleaning a body cavity.

FIG. 12 illustrates a method for constructing a cleansing device.

### DETAILED DESCRIPTION OF THE INVENTION

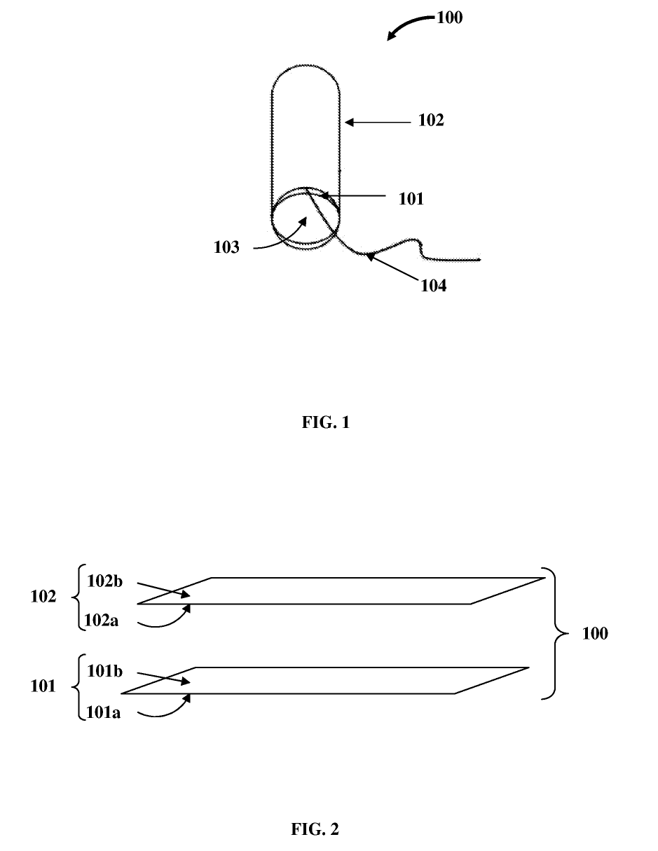

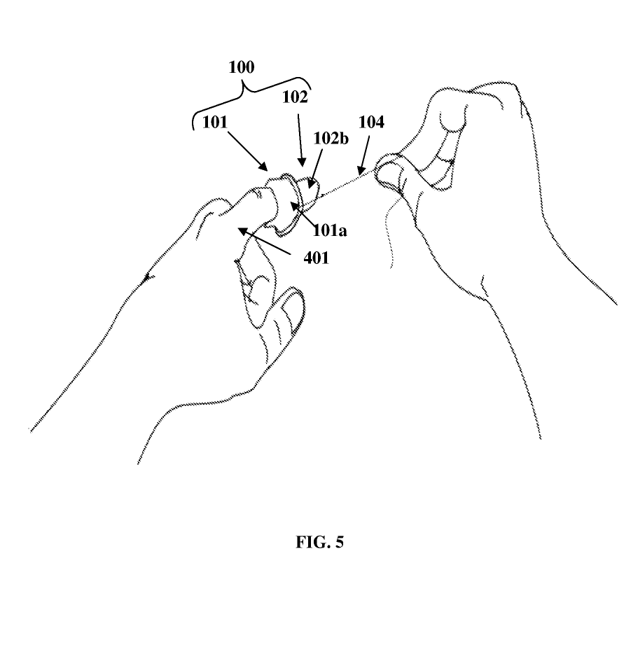

FIG. 1 exemplarily illustrates a cleansing device 100 for cleaning a body cavity 402. As used herein, the term “body cavity” refers to a tubular region or any internal body area, for example, a vaginal canal, a nasal cavity, an oral cavity, a rectal cavity, etc. in the body accessible from an external orifice. For purposes of illustration, the body cavity 402 referred to herein is a vaginal canal as exemplarily illustrated in FIG. 4, which is also referenced by the numeral 402. The cleansing device 100 disclosed herein comprises an inner membrane 101 and an outer membrane 102. The inner membrane 101 defines an inner surface 101a and an outer surface 101b, and the outer membrane 102 defines an inner surface 102a and an outer surface 102b as disclosed in the detailed description of FIG. 2. The inner surface 101a of the inner membrane 101 defines a cavity 103 configured to conform to and accommodate a finger 401, for example, an index finger, of a user. As used herein, the term “user” refers to a person who uses the cleansing device 100 to clean a body cavity 402, for example, a vaginal canal, a nasal cavity, an oral cavity, a rectal cavity, etc. The inner membrane 101 is in contact with and is enveloped by the outer membrane 102 to form the cleansing device 100.

The outer membrane 102 of the cleansing device 100 is fabricated from a non-bulky hypoallergenic material. The term “hypoallergenic material” refers to a material that produces minimal or no allergenic reactions. The hypoallergenic material is, for example, cotton, microfiber, feather down, etc. The inner membrane 101 is fabricated from an elastomeric material. The elastomeric material is, for example, nitrile, latex, cyanamidite, peridine, amine, etc., and any combination thereof. In an embodiment, the cleansing device 100 further comprises a string 104 attached between the inner membrane 101 and the outer membrane 102, for example, using an adhesive, for enabling removal of the cleansing device 100 from the user's finger 401. The string 104 is made of a hypoallergenic material, for example, cotton.

FIG. 2 exemplarily illustrates the inner membrane 101 and the outer membrane 102 used for constructing the cleansing device 100 disclosed herein. The inner membrane 101 defines an inner surface 101a and an outer surface 101b. The outer membrane 102 also defines an inner surface 102a and an outer surface 102b. The inner surface 101a of the inner membrane 101 defines a cavity 103 configured to conform to and accommodate the user's finger 401. The inner membrane 101 is in contact with and enveloped by the outer membrane 102 to form the cleansing device 100. When the user inserts the finger 401 into the cavity 103 of the cleansing device 100, the inner surface 101a of the inner membrane 101 contacts the user's finger 401, and the outer surface 102b of the outer membrane 102 is exposed for cleaning the body cavity 402. In an embodiment, the inner surface 101a of the inner membrane 101 is configured for gripping the user's finger 401. For example, the inner surface 101a of the inner membrane 101 is made of a tacky or textured material that makes adhesive contact with the user's finger 401. In an embodiment, the user may also roll the cleansing device 100 disclosed herein onto a finger 401.

For the purposes of illustration, the detailed description refers to an inner membrane 101 and an outer membrane 102; however, the scope of the cleansing device 100 disclosed herein is not limited to the inner membrane 101 and the outer membrane 102 but may be extended to include a single layer or multiple layers comprising one or more membranes.

The string 104 of the cleansing device 100 disclosed herein, as exemplarily illustrated in FIG. 1, is attached between the outer surface 101b of the inner membrane 101 and the inner surface 102a of the outer membrane 102, such that a part of the string 104 is within the cleansing device 100 and the remaining part of the string 104 extends out from the cleansing device 100. In an example, a part of the string 104 is attached between the outer surface 101b of the inner membrane 101 and the inner surface 102a of the outer membrane 102, for example, using an adhesive and the remaining part of the string 104 extends out from the cleansing device 100. The string 104 is attached to both the outer surface 101b of the inner membrane 101 and the inner surface 102a of the outer membrane 102, for example, by a stitch, an adhesive, or other suitable fastening element.

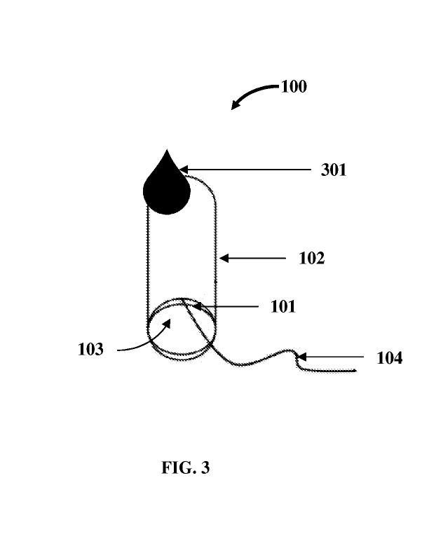

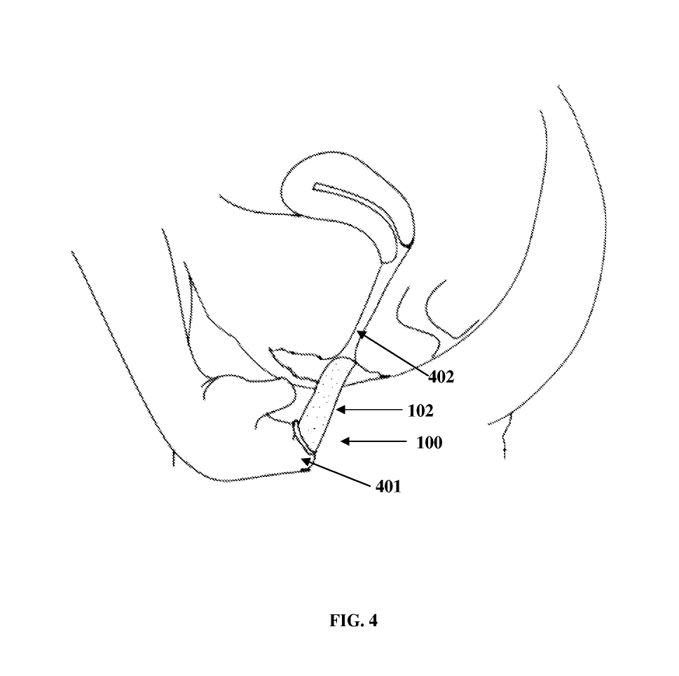

FIG. 3 exemplarily illustrates a soakable cleansing device 100. The cleansing device 100 disclosed herein can be soaked in a sterile solution 301, for example, water, for improving grip between the user's finger 401 and the cleansing device 100. The soaked cleansing device 100 grips the user's finger 401 and prevents slippage of the cleansing device 100 from the user's finger 401 during the cleaning of the body cavity 402. In an example, the user inserts the finger 401 into the cavity 103 of the cleansing device 100 and then immerses the finger 401 enclosed by the cleansing device 100 into a glass of sterile water, thereby soaking the cleansing device 100. On soaking, the cleansing device 100 immediately conforms and clings to the user's finger 401, thereby reducing the chance of any slippage of the cleansing device 100 from the user's finger 401.

FIG. 4 exemplarily illustrates introduction of a user's finger 401 enclosed by the cleansing device 100 into a body cavity 402. The body cavity 402 is herein referred to as a “vaginal canal” of a female user. For cleaning the vaginal canal 402, the user inserts a finger 401, for example, an index finger, a middle finger, etc., into the cavity 103 of the cleansing device 100. The user then introduces the finger 401 enclosed by the cleansing device 100 into the vaginal canal 402 through an external orifice of the vaginal canal 402 for cleaning the vaginal canal 402. The user cleanses the vaginal canal 402 by moving the enclosed finger 401 in horizontal, vertical, and circular directions along an inner wall of the vaginal canal 402.

FIG. 5 exemplarily illustrates removal of the cleansing device 100 from the user's finger 401 by the string 104. For removing the cleansing device 100 from the user's finger 401, the user holds the part of the string 104 that extends out from the cleansing device 100 and pulls the string 104 outwardly. Removal of the cleansing device 100 from the user's finger 401 exposes the inner membrane 101 of the cleansing device 100. The outward pull of the string 104 removes the cleansing device 100 from the user's finger 401, whereby the outer surface 102b of the outer membrane 102 that cleans the body cavity 402 folds inwardly, and the inner surface 101a of the inner membrane 101 is exposed outwardly for ensuring safe and hygienic disposal of the cleansing device 100.

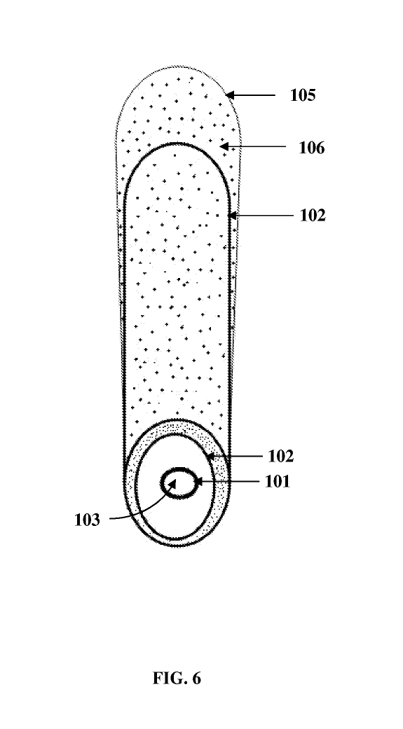

FIG. 6 exemplarily illustrates an embodiment of the cleansing device 100, showing a covering member 105 covering the outer membrane 102 of the cleansing device 100. The covering member 105 contains one or more permeable fluids 106 and covers the outer surface 102b of the outer membrane 102 with the permeable fluids 106. The covering member 105 releases the permeable fluids 106 over the outer surface 102b of the outer membrane 102. A user inserts a finger 401 into the cavity 103 of the cleansing device 100 and inserts the covering member 105 containing the permeable fluids 106 over the outer membrane 102 of the cleansing device 100. The permeable fluids 106 contained in the covering member 105 permeate out of the covering member 105, and into the outer surface 102b of the outer membrane 102 of the cleansing device 100. When the outer surface 102b of the outer membrane 102 of the cleansing device 100 is covered with the permeable fluids 106, the user removes the covering member 105 from the finger 401 enclosed by the cleansing device 100. The user then cleanses the body cavity 402 with the outer membrane 102 of the cleansing device 100 covered with the permeable fluids 106.

In an embodiment, the permeable fluids 106 comprise a medicating element, for example, anti-infectives, anti-inflammatories, coronary vasodilators, anesthetics, anti-tussives, expectorants, estrogenic, progestational or prostaglandin agents, etc. The medicating element is used to treat a condition, for example, an infection, in the body cavity 402. In another embodiment, the permeable fluids 106 comprise a germicide, for example, antibiotics, disinfectants, etc., for killing germs and bacteria in the body cavity 402.

In another embodiment, the permeable fluids 106 comprise a perfuming element. The term “perfuming element” refers to a substance that emits and diffuses a pleasant odor. The perfuming element is, for example, a deodorant, a scenting compound, etc. In an example, the covering member 105 is filled with a permeable deodorant. The user inserts a finger 401 into the cavity 103 of the cleansing device 100 and inserts the covering member 105 filled with the permeable deodorant over the outer membrane 102 of the cleansing device 100. The permeable deodorant contained within the covering member 105 permeates into the outer surface 102b of the outer membrane 102 of the cleansing device 100. The user then removes the covering member 105 from the cleansing device 100, and inserts the finger 401 enclosed by the cleansing device 100 into a vaginal canal 402. When the user moves the enclosed finger 401 against the inner wall of the vaginal canal 402, the permeable deodorant from the outer surface 102b of the outer membrane 102 releases into the vaginal canal 402 and replaces any unpleasant odor by a pleasing odor.

In another embodiment, the permeable fluids 106 comprise a lubricant, for example, a permeable gel. The term “lubricant” refers to a substance introduced between two surfaces to reduce the friction between the surfaces. In an example, the covering member 105 is filled with a permeable lubricant. The user inserts a finger 401 into the cavity 103 of the cleansing device 100 and inserts the covering member 105 filled with the permeable lubricant over the outer membrane 102 of the cleansing device 100. The permeable lubricant contained within the covering member 105 permeates into the outer surface 102b of the outer membrane 102 of the cleansing device 100. The user then removes the covering member 105 from the cleansing device 100, and inserts the finger 401 enclosed by the cleansing device 100 into the vaginal canal 402. The lubricant from the outer surface 102b of the outer membrane 102 releases into the vaginal canal 402 and reduces friction between the outer surface 102b of the outer membrane 102 of the cleansing device 100 and the inner wall of the vaginal canal 402, thereby increasing efficiency of cleaning and reducing discomfort for the user.

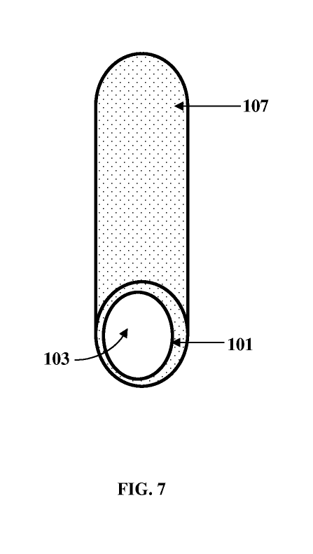

FIG. 7 exemplarily illustrates an embodiment of the cleansing device 100, showing a moisture laden outer membrane 107 of the cleansing device 100. The outer membrane 102 of the cleansing device 100 is soaked in a sterile solution 301, for example, water, for a period of time to allow the outer membrane 102 to absorb and contain the water or moisture, thereby forming the moisture laden outer membrane 107. When the user inserts a finger 401 into the cavity 103 defined by the inner surface 101a of the inner membrane 101 of the cleansing device 100 and introduces the finger 401 enclosed by the cleansing device 100 into the body cavity 402 for cleaning the body cavity 402, the moisture laden outer membrane 107 provides smooth contact between the outer surface 102b of the moisture laden outer membrane 107 and the body cavity 402. The moisture laden outer membrane 107 reduces friction and provides a smooth outer surface 102b for cleaning the body cavity 402, thereby decreasing discomfort for the user.

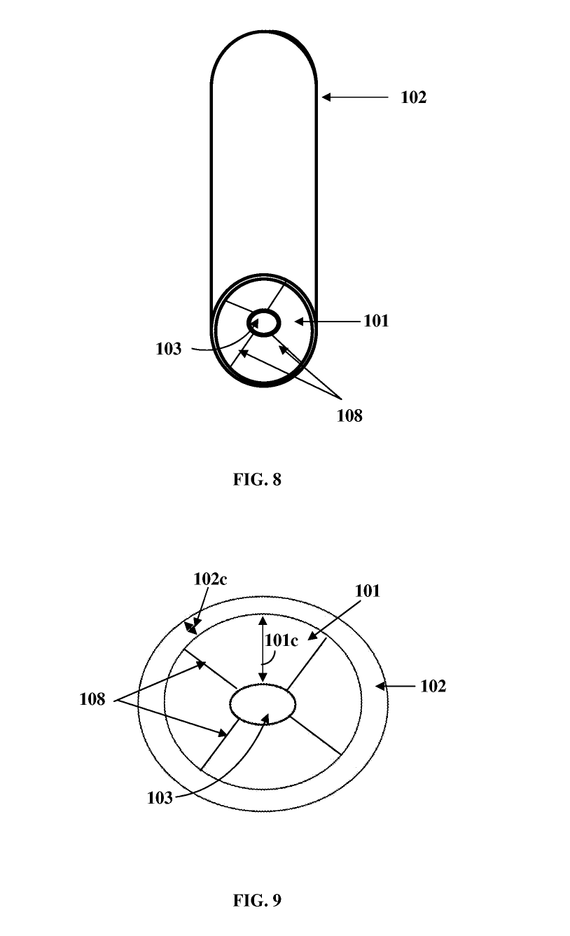

FIG. 8 exemplarily illustrates an embodiment of the cleansing device 100, showing the configuration of the inner membrane 101 of the cleansing device 100 for improving grip of the cleansing device 100 on the user's finger 401. The inner surface 101a of the inner membrane 101 is configured to provide additional grip between the cleansing device 100 and the user's finger 401. The additional grip provided prevents slippage of the cleansing device 100 from the user's finger 401. In an embodiment, the inner surface 101a of the inner membrane 101 has a ribbed structure 108 that provides additional grip between the user's finger 401 and the cleansing device 100.

FIG. 9 exemplarily illustrates an embodiment of the cleansing device 100, showing the thicknesses 101c and 102c of the inner membrane 101 and the outer membrane 102 respectively of the cleansing device 100 for improving grip of the cleansing device 100 on the user's finger 401. The material of the inner membrane 101 of the cleansing device 100 and the material of the outer membrane 102 of the cleansing device 100 is configured with sufficient thicknesses 101c and 102c respectively, to ensure a firm grip of the cleansing device 100 on the user's finger 401. When the user's finger 401 is inserted into the cleansing device 100, the thick materials firmly grip the user's finger 401, thereby preventing slippage of the cleansing device 100 from the user's finger 401 during cleaning of the body cavity 402.

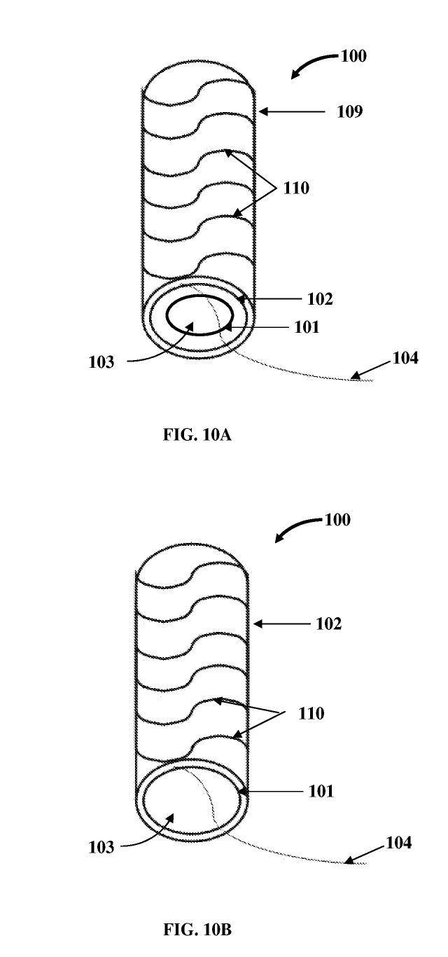

FIG. 10A exemplarily illustrates an embodiment of the cleansing device 100, showing a pleated layer 109 enveloping the outer membrane 102 of the cleansing device 100. The pleated layer 109 comprises multiple pleats 110 made of an adsorbent material, for example, a sponge, etc., that contains a sanitizing fluid, for example, a sanitizing solution, a germicide, a medicating fluid, etc., for disinfecting and cleaning the body cavity 402. The pleated layer 109 allows better sanitization of the body cavity 402 than a non-pleated surface. Moreover, the pleated layer 109 better conforms to the inner wall of the body cavity 402. Furthermore, the pleated layer 109 flexes and yields to reduce friction, abrasion, and inadvertent pressure against the inner wall of the body cavity 402 as the enclosed finger 401 is maneuvered inside the body cavity 402.

FIG. 10B exemplarily illustrates an embodiment of the cleansing device 100, showing the outer membrane 102 of the cleansing device 100 configured with pleats 110. The pleats 110 are made of an adsorbent material, for example, a sponge, etc., that contains a sanitizing fluid, for example, a sanitizing solution, a germicide, a medicating fluid, etc., for disinfecting and cleaning the body cavity 402.

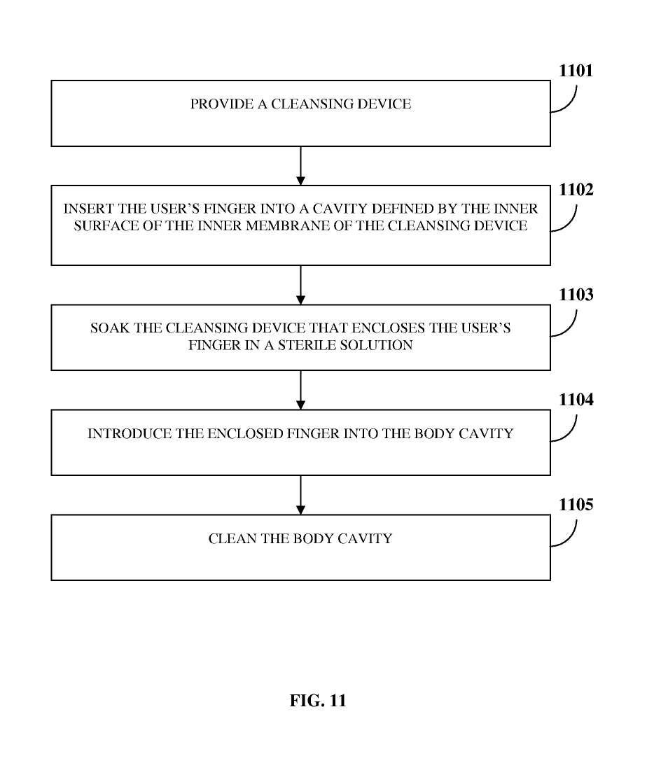

FIG. 11 exemplarily illustrates a method for cleaning a body cavity 402. A cleansing device 100 as disclosed in the detailed description of FIGS. 1-3 is provided 1101. The inner surface 101a of the inner membrane 101 defines a cavity 103 configured to conform to and accommodate a user's finger 401. The user inserts 1102 a finger 401 into the cavity 103 of the cleansing device 100. The cleansing device 100 encloses the user's finger 401. The user's finger 401 is inserted in the cavity 103, where the inner surface 101a of the inner membrane 101 contacts the user's finger 401, and the outer surface 102b of the outer membrane 102 is exposed for cleaning the body cavity 402. The user then introduces 1104 the finger 401 enclosed by the cleansing device 100 into the body cavity 402 as exemplarily illustrated in FIG. 4. In an embodiment, the cleansing device 100 is soaked 1103 in a sterile solution 301, for example, water, as exemplarily illustrated in FIG. 3, prior to introduction of the enclosed finger 401 into the body cavity 402 for improving the grip between the user's finger 401 and the cleansing device 100. The user cleans 1105 the body cavity 402 using the exposed outer surface 102b of the outer membrane 102 by moving the finger 401 enclosed by the cleansing device 100 in horizontal, vertical, and circular directions along the inner wall of the body cavity 402.

In an embodiment, the user's finger 401 enclosed by the cleansing device 100 is retracted from the body cavity 402, immersed in a sterile solution 301 for rinsing the cleansing device 100, and reintroduced into the body cavity 402. The above process is repeated, until the body cavity 402 is completely cleansed and refreshed. The user removes the cleansing device 100 from the finger 401 by outwardly pulling the string 104 attached between the outer surface 101b of the inner membrane 101 and the inner surface 102a of the outer membrane 102 as exemplarily illustrated in FIG. 5.

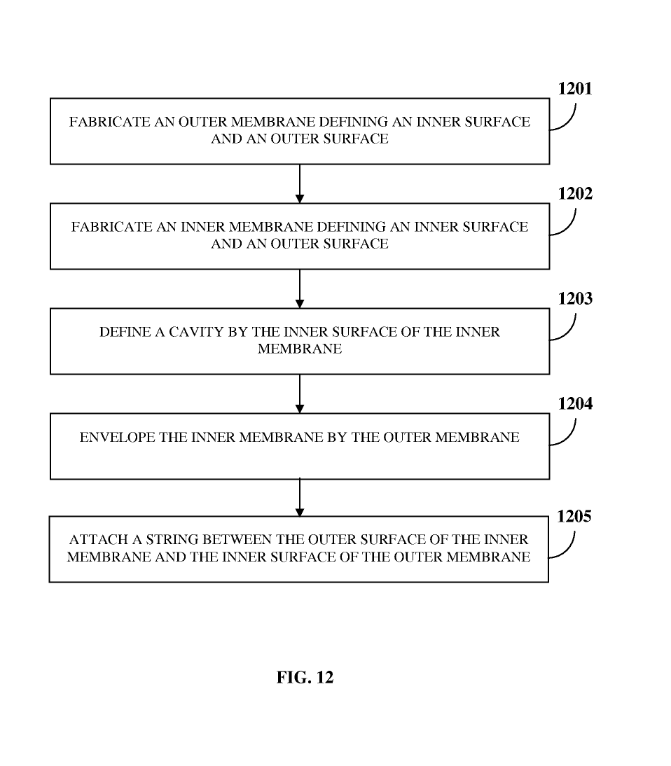

FIG. 12 illustrates a method for constructing a cleansing device 100. An outer membrane 102 defining an inner surface 102a and an outer surface 102b is fabricated 1201 from a hypoallergenic material, for example, cotton, micro fiber, feather down, etc. An inner membrane 101 defining an inner surface 101a and an outer surface 101b is fabricated 1202 from an elastomeric material, for example, nitrile, latex, cyanamidite, peridine, amine, etc., and any combination thereof. The inner surface 101a of the inner membrane 101 defines 1203 a cavity 103 configured to conform to and accommodate a user's finger 401. The inner membrane 101 is enveloped 1204 by the outer membrane 102, where the outer surface 101b of the inner membrane 101 contacts the inner surface 102a of the outer membrane 102 to form the cleansing device 100 as exemplarily illustrated in FIG. 1. The inner surface 101a of the inner membrane 101 contacts the user's finger 401 when the user inserts the finger 401 in the cavity 103 of the cleansing device 100. In an embodiment, a string 104 made of a hypoallergenic material, for example, cotton, is attached 1205 between the outer surface 101b of the inner membrane 101 and the inner surface 102a of the outer membrane 102, for example, by a stitch, an adhesive, etc., for enabling removal of the cleansing device 100 from the user's finger 401. The cleansing device 100 encloses the finger 401 inserted in the cavity 103 defined by the cleansing device 100. The user then introduces the enclosed finger 401 into the body cavity 402 for cleaning the body cavity 402.

In an embodiment, a covering member 105 containing one or more permeable fluids 106 covers the outer surface 102b of the outer membrane 102 as disclosed in the detailed description of FIG. 6. In another embodiment, the outer membrane 102 of the cleansing device 100 is enveloped with a pleated layer 109 or configured with pleats 110 that contain a sanitizing fluid for disinfecting and cleaning the body cavity 402 as disclosed in the detailed description of FIGS. 10A-10B.

Consider an example where a female user utilizes the cleansing device 100 disclosed herein for cleaning her vaginal canal 402. The female user inserts her finger 401 into the cavity 103 of the cleansing device 100 and soaks her finger 401 enclosed by the cleansing device 100 in a sterile solution 301, for example, water. The female user then introduces her enclosed finger 401 into her vaginal canal 402. She cleans her vaginal canal 402 and removes, for example, foreign and natural substances from her vaginal canal 402 by moving her enclosed finger 401 in horizontal, vertical, and circular directions along the inner wall of her vaginal canal 402. The female user then rinses the cleansing device 100 by retracting her enclosed finger 401 from her vaginal canal 402 and immersing her enclosed finger 401 in a glass of sterile water. After rinsing the cleansing device 100, the female user reintroduces her rinsed and enclosed finger 401 into her vaginal canal 402. She repeats the above stated process until her vaginal canal 402 is cleaned as desired. The female user can use the cleansing device 100 disclosed herein for cleaning her vaginal canal 402, for example, before, during, and after menstruation. The female user can use the cleansing device 100 disclosed herein for cleaning the vaginal canal 402 up to and around the cervix.

The foregoing examples have been provided merely for the purpose of explanation and are in no way to be construed as limiting of the invention disclosed herein. While the invention has been described with reference to various embodiments, it is understood that the words, which have been used herein, are words of description and illustration, rather than words of limitation. Further, although the invention has been described herein with reference to particular means, materials and embodiments, the invention is not intended to be limited to the particulars disclosed herein; rather, the invention extends to all functionally equivalent structures, methods and uses, such as are within the scope of the appended claims. Those skilled in the art, having the benefit of the teachings of this specification, may effect numerous modifications thereto and changes may be made without departing from the scope and spirit of the invention in its aspects.

I claim:

1. A device for cleaning a body cavity, comprising:
an outer membrane defining an inner surface and an outer surface;
an inner membrane defining an inner surface and an outer surface, wherein said inner surface of said inner membrane defines a cavity configured to conform to and accommodate a finger of a user; and
said inner membrane in contact with and enveloped by said outer membrane to form said device;
whereby said finger is inserted into said cavity of said device and said finger enclosed by said device is introduced into said body cavity for cleaning said body cavity.

2. The device of claim 1, wherein said outer membrane is fabricated from a hypoallergenic material.

3. The device of claim 2, wherein said hypoallergenic material is one of cotton, microfiber, and feather down.

4. The device of claim 1, wherein said inner membrane is fabricated from an elastomeric material.

5. The device of claim 4, wherein said elastomeric material is one of nitrile, latex, cyanamidite, peridine, amine, and any combination thereof.

6. The device of claim 1, further comprising a string attached between said outer surface of said inner membrane and said inner surface of said outer membrane for enabling removal of said device from said finger of said user.

7. The device of claim 6, wherein said string is made of a hypoallergenic material.

8. The device of claim 1 soaked in a sterile solution, wherein said soaked device that encloses said finger of said user grips said finger of said user and prevents slippage of said device from said finger of said user during said cleaning of said body cavity.

9. The device of claim 1, wherein said inner surface of said inner membrane is configured for gripping said finger of said user.

10. The device of claim 1, further comprising a covering member that covers said outer membrane, wherein said covering member contains one or more permeable fluids and releases said one or more permeable fluids over said outer surface of said outer membrane.

11. The device of claim 10, wherein said one or more permeable fluids contained in said covering member are one or more of a medicating element, a germicide, a perfuming element, a lubricant, and any combination thereof.

12. The device of claim 1, wherein said outer membrane is a moisture laden outer membrane for providing smooth contact between said outer surface of said outer membrane and said body cavity.

13. The device of claim 1, further comprising a pleated layer enveloping said outer membrane of said device.

14. The device of claim 13, wherein said pleated layer is made of an adsorbent material that contains a sanitizing fluid for disinfecting and cleaning said body cavity.

15. The device of claim 1, wherein said outer membrane is configured with pleats that contain a sanitizing fluid for disinfecting and cleaning said body cavity.

16. A method for cleaning a body cavity of a user, comprising:
providing a cleansing device comprising:
an outer membrane defining an inner surface and an outer surface;
an inner membrane defining an inner surface and an outer surface, wherein said inner surface of said inner membrane defines a cavity configured to conform to and accommodate a finger of a user; and
said inner membrane in contact with and enveloped by said outer membrane to form said cleansing device;
inserting said finger of said user into said cavity of said cleansing device and enclosing said finger of said user by said cleansing device, wherein said inner surface of said inner membrane contacts said finger of said user, and said outer surface of said outer membrane is exposed for cleaning said body cavity;
introducing said enclosed finger of said user into said body cavity; and
cleaning said body cavity using said exposed outer surface of said outer membrane of said cleansing device by moving said enclosed finger of said user in horizontal, vertical, and circular directions along an inner wall of said body cavity.

17. The method of claim 16, further comprising soaking said cleansing device in a sterile solution prior to said introduction of said enclosed finger of said user into said body cavity, wherein said soaked cleansing device that encloses said finger of said user grips said finger of said user and prevents slippage of said cleansing device from said finger of said user during said cleaning of said body cavity.

18. The method of claim 16, further comprising removing said cleansing device from said finger of said user by outwardly pulling a string attached between said outer surface of said inner membrane and said inner surface of said outer membrane, wherein said outward pull of said string removes said cleansing device from said finger of said user, whereby said outer surface of said outer membrane that cleans said body cavity folds inwardly, and said inner surface of said inner membrane is exposed outwardly for ensuring safe and hygienic disposal of said cleansing device.

19. The method of claim 16, further comprising rinsing said cleansing device in a sterile solution after retraction of said enclosed finger of said user from said body cavity and prior to reintroduction of said enclosed finger of said user into said body cavity.

20. A method for constructing a cleansing device for cleaning a body cavity of a user, comprising:
fabricating an outer membrane defining an inner surface and an outer surface;
fabricating an inner membrane defining an inner surface and an outer surface;
defining a cavity configured to conform to and accommodate said finger of said user by said inner surface of said inner membrane; and
enveloping said inner membrane by said outer membrane, wherein said outer surface of said inner membrane contacts said inner surface of said outer membrane to form said cleansing device;
whereby said cleansing device encloses said finger inserted in said cavity, and said user introduces said enclosed finger into said body cavity for cleaning said body cavity.

21. The method of claim 20, wherein said outer membrane is fabricated from a hypoallergenic material, wherein said hypoallergenic material is one of cotton, microfiber, and feather down.

22. The method of claim 20, wherein said inner membrane is fabricated from an elastomeric material, wherein said elastomeric material is one of nitrile, latex, cyanamidite, peridine, amine, and any combination thereof.

23. The method of claim 20, further comprising attaching a string between said outer surface of said inner membrane and said inner surface of said outer membrane for enabling removal of said cleansing device from said finger of said user, wherein said string is made of a hypoallergenic material.

24. The method of claim 20, further comprising covering said outer membrane of said cleansing device with a covering member, wherein said covering member contains one or more permeable fluids and releases said one or more permeable fluids over said outer surface of said outer membrane, wherein said one or more permeable fluids are one or more of a medicating element, a germicide, a perfuming element, a lubricant, and any combination thereof.

25. The method of claim 20, further comprising one of:
enveloping said outer membrane with a pleated layer made of an adsorbent material that contains a sanitizing fluid for disinfecting and cleaning said body cavity; and
configuring said outer membrane with pleats that contain said sanitizing fluid for disinfecting and cleaning said body cavity.
